(19) United States
(12) Patent Application Publication
(10) Pub. No.: US 2008/0095844 A1
(43) Pub. Date: Apr. 24, 2008

(54) SUSTAINED RELEASE PHARMACEUTICAL COMPOSITIONS OF ALFUZOSIN AND PROCESS FOR PREPARATION THEREOF

(76) Inventors: Rajhans Sujay Kamalakar, Gujarat (IN); Bhutada Pravin Meghraj, Gujarat (IN); Patel Hasmukh Mathurbhai, Gujarat (IN)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/841,148

(22) Filed: Aug. 20, 2007

(30) Foreign Application Priority Data
Oct. 23, 2006 (IN) 1758/MUM/2006

(51) Int. Cl. A61K 9/22; A61K 31/517; A61K 9/20

(52) U.S. Cl. 424/465; 424/468; 514/266.24

(57) ABSTRACT

The present invention relates to sustained release pharmaceutical compositions comprising alfuzosin, a rate-controlling polymer and optionally one or more pharmaceutically acceptable excipients; process for preparing such compositions and method of using the compositions.

### CROSS-REFERENCE TO RELATED APPLICATION

This application claims priority to Indian Provisional Application Number 1758/MUM/2006, filed on Oct. 23, 2006, the entire disclosure of which is herein incorporated in its entirety.

### FIELD OF THE INVENTION

The present invention relates to sustained release pharmaceutical compositions of alfuzosin, process for preparing such compositions and method of using such compositions.

### BACKGROUND OF THE INVENTION

Alfuzosin is a selective ∝₁ adrenoceptor antagonist that belongs to the chemical class of 4-amino-6,7-dimethoxy quinazol-2-yl-alkylene diamines. Alfuzosin acts as a selective and competitive antagonist of α₁-adrenoceptor mediated contraction of prostatic, prostatic capsule, bladder base and proximal urethral structures and is used in the treatment of signs and symptoms of benign prostatic hyperplasia.

Alfuzosin has a short half-life and shows the characteristic of being absorbed preferentially in the upper part of the gastrointestinal tract and, in particular, being absorbed in the duodenum and the jejunum. Sustained release compositions of alfuzosin provide various advantages over conventional multiple dosing including better patient compliance, reduced fluctuations of plasma drug levels, and reduced toxicity.

U.S. Pat. No. 6,149,940 discloses a preparation of an alfuzosin once daily composition for oral delivery using a Geomatrix technology that has been developed by Jagotec-AG. The three-layer Geomatrix tablet described in this patent consists of a hydrophilic active matrix core containing alfuzosin hydrochloride and two inert, functional layers (one swellable layer and one erodible layer) whose function is to control the hydration and swelling rate of the core, and thereby slow down and linearize the dissolution of the drug. When the tablet comes into contact with gastric juices, it increases considerably in volume and thus remains in the stomach for a longer time. In this manner, most of the drug is absorbed in a controlled manner in the portion of the gastrointestinal tract having better absorption window. The alfuzosin is released in zero order from the dosage form developed using this technology.

The WO 97/18814 patent application assigned to Pfizer discloses a controlled-release pharmaceutical formulation of doxazosin with low molecular weight polyethylene oxide and hydroxypropylmethyl cellulose.

U.S. Patent Applications No. 2006/0062846 and 2006/0062845 assigned to Cimex disclose a monolithic composition of alfuzosin which does not float in gastric fluid. The applications disclose compositions which show desired dissolution profile with more than 70% by weight of hydroxypropyl methylcellulose based on the weight of the total composition. The applications also disclose use of polyvinylpyrrolidone as a dry binder in the composition.

The WO 04/37228 patent application assigned to Ranbaxy discloses a sustained release oral dosage form that includes a single functional layer and, optionally, one or more nonfunctional layers adjacent to the single functional layer. The single functional layer includes alfuzosin or pharmaceutically acceptable salt, solvate, enantiomers or mixtures thereof and one or more release retarding ingredients. The release retarding ingredient may be one or more of cellulose polymer, methacrylate polymer, acrylic acid polymer, block copolymer, gum or polyethylene oxide. It discloses use of polyvinylpyrrolidone as a dry binder.

The WO 2006/94736 patent application discloses controlled release tablets of alfuzosin hydrochloride containing different grades of hydroxypropyl methylcellulose, 1-4% of polyvinylpyrrolidone and lactose.

It was observed that preparation of sustained release alfuzosin formulation which is bioequivalent to the reference product available in various countries, specifically UroXatral® marketed by Sanofi in United States, is difficult due to unique nature of the formulation and specific absorption window of the drug in GI tract. Efforts to prepare bioequivalent composition using teachings of the prior art have resulted in failure.

We have surprisingly found that the sustained release pharmaceutical composition of alfuzosin can be prepared by incorporating a mixture of two or more polymers only in the specific concentration in the composition.

### SUMMARY OF THE INVENTION

In one aspect, the invention provides a sustained release pharmaceutical composition comprising:
(i) alfuzosin,
(ii) a rate controlling polymer, and
(iii) optionally one or more pharmaceutically acceptable excipients.

In another aspect, the invention provides a sustained release pharmaceutical composition comprising:
(i) alfuzosin,
(ii) a rate controlling polymer, and
(iii) optionally one or more pharmaceutically acceptable excipients, wherein the rate controlling polymer is present extragranular.

In another aspect, the invention provides a process for preparation of a sustained release pharmaceutical composition, wherein the process comprises:
(i) mixing alfuzosin, a rate controlling polymer and optionally one or more pharmaceutically acceptable excipients,
(ii) granulating the mixture of step (i) with a granulating solvent or a solution,
(iii) drying the granules of step (ii),
(iv) mixing the granules of step (iii) with a rate controlling polymer and optionally one or more pharmaceutically acceptable excipients, and
(v) compressing the mixture of step (iv) into a tablet.

In another aspect, the invention provides a process for preparation of a sustained release pharmaceutical composition, wherein the process comprises:
(i) mixing alfuzosin with optionally one or more pharmaceutically acceptable excipients,
(ii) granulating the mixture of step (i) with a granulating solvent or a solution,
(iii) drying the granules of step (ii),
(iv) mixing the granules of step (iii) with a rate controlling polymer and optionally one or more pharmaceutically acceptable excipients, and
(v) compressing the mixture of step (iv) into a tablet.

In another aspect, the invention discloses a process for preparation of a sustained release pharmaceutical composition, wherein the process comprises:
(i) mixing alfuzosin, a rate controlling polymer and optionally one or more pharmaceutically acceptable excipients,
(ii) granulating the mixture of step (i) with a granulating solvent or a solution,
(iii) drying the granules of step (ii),
(iv) mixing the granules of step (iii) with optionally one or more pharmaceutically acceptable excipients, and
(v) compressing the mixture of step (iv) into a tablet.

In yet another aspect, the invention provides sustained release pharmaceutical composition consisting essentially of:
(i) 1-15% by weight alfuzosin,
(ii) 35-69% by weight hydroxypropyl methylcellulose,
(iii) 10-20% by weight polyvinylpyrrolidone, and
(iv) 5-30% by weight lactose.

In further aspect, the invention provides a method for the treatment of the signs and symptoms of benign prostatic hyperplasia, wherein the method comprises administering to a patient in need thereof a sustained release pharmaceutical composition of alfuzosin.

These and other features, advantages and objects of the present invention will be further understood and appreciated by those skilled in the art by reference to the following specification and claims.

### DETAILED DESCRIPTION OF THE INVENTION

The term “alfuzosin” as used herein refers to alfuzosin free base or pharmaceutically acceptable salts, hydrates, solvates and enantiomers thereof or mixtures thereof. The preferred salt of alfuzosin is alfuzosin hydrochloride. Alfuzosin may be present in an amount ranging from 1% to 20% by weight of the composition.

The term “sustained release pharmaceutical composition” as used herein is intended for a composition which provides the desired therapeutic effect of alfuzosin for a period of more than 12 hours, or for a period of 24 hours.

The term “rate controlling polymer” as used herein and appended claims refers to a mixture of polyvinylpyrrolidone and one or more hydrophilic polymers other than polyvinylpyrrolidone such that polyvinylpyrrolidone is present in an amount of more than 8% by weight of the composition and the hydrophilic polymer is present in an amount ranging from 35 to 69% by weight of the composition. Suitable hydrophilic polymers may be hydroxypropyl methylcellulose, hydroxypropyl cellulose, hydroxyethyl cellulose, hydroxypropyl ethylcellulose, methyl cellulose, carboxymethylcellulose, sodium alginate, xanthan gum, locust bean gum, alginic acid, methacrylate copolymer or mixtures thereof. The rate controlling polymer may be present intragranularly, extragranularly or both. The rate controlling polymer provides the sustained release of alfuzosin for a period of more than 12 hours, or for a period of 24 hours. The rate controlling polymer may comprise polyvinylpyrrolidone in an amount ranging from 10% to 20% by weight of the composition. Polyvinylpyrrolidone (PVP) may be present intragranularly, extragranularly or both. Polyvinylpyrrolidone may be present in an amount of less than 5% by weight intragranularly, and more than 5% by weight extragranularly. Polyvinylpyrrolidone may be selected from different grades available such as low molecular weight PVP (molecular weight less than or equal to 50,000 daltons) and high molecular weight PVP (molecular weight more than 50,000 daltons). Low molecular weight PVP may be present intragranularly and high molecular weight PVP may be present extragranularly. PVP may act as a binder and/or a rate controlling polymer depending on its concentration. For example, when compositions contain intragranular polyvinylpyrrolidone in an amount of less than 5% by weight of the composition and extragranular polyvinylpyrrolidone in an amount of more than 5% by weight of the composition, the intragranular PVP may act as a binder and extragranular PVP may act as a rate controlling polymer. Similarly for example, when compositions contain intragranular polyvinylpyrrolidone in an amount of 4.5% by weight of the composition and extragranular polyvinylpyrrolidone in an amount of 4.5% by weight of the composition, the intragranular PVP may act as a binder and extragranular PVP may act as a rate controlling polymer. When compositions contain more than 8% by weight of polyvinylpyrrolidone, irrespective of the amount distributed intragranular or extragranular, the total amount of polyvinylpyrrolidone may act as rate controlling polymer when present with other hydrophilic polymer in the range of 35-69% by weight of the composition.

The pharmaceutical compositions as described herein may comprise one or more pharmaceutically acceptable excipients selected from diluent, binder, disintegrant, lubricant and mixtures thereof.

Diluent may be selected from powdered cellulose, microcrystalline cellulose, silicified microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol or erythritol; and mixtures thereof. The diluent may be present in an amount ranging from 5% to 80% by weight of the composition.

Binder may be selected from hydroxyethyl cellulose, hydroxypropyl cellulose, hydroxypropyl methylcellulose, carbomers, dextrin, ethyl cellulose, methylcellulose, gelatin, polymethacrylates, polyvinylpyrrolidone, pregelatinized starch, sodium alginate, gums, synthetic resins and the like. The binder may be present in an amount ranging from 0.1% to 8% by weight of the composition.

Disintegrant may be selected from croscarmellose sodium, sodium starch glycolate, pregelatinized starch, sodium carboxymethyl cellulose, microcrystalline cellulose, cross-linked polyvinylpyrrolidone and mixtures thereof. The disintegrant may be present in an amount ranging from 1% to 10% by weight of the composition.

Lubricant/glidant may be selected from talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, sodium stearyl fumarate, sodium benzoate, mineral oil, magnesium trisilicate; and mixtures thereof. The lubricant/glidant may be present in an amount ranging from 0.1% to 8% by weight of the composition.

A granulating solvent may be selected from water, isopropyl alcohol, ethanol, methanol, acetone, methylene chloride or mixtures thereof. The granulating solution may comprise any binder or mixture of binders in the granulating solvent.

The sustained release pharmaceutical composition of alfuzosin as described herein exhibits a dissolution of not more than 30% in 4 hour, not more than 45% in 8 hours, not more than 60% in 12 hours, not more than 75% in 20 hours, as measured in 900 ml of 0.01 N HCl using USP Type II apparatus with a paddle speed of 50 rpm at 37±0.5° C.

The pharmaceutical composition may be coated using suitable coating techniques known in the art which do not affect the dissolution properties of the final formulation. The film coat may comprise film-forming polymer such as hydroxypropyl methylcellulose, hydroxyethylcellulose, sodium carboxymethylcellulose, polyvinyl pyrrolidone, and the like; a plasticizer such as glyceryltriacetate, dibutyl sebacate, diethylphthalate, polyethylene glycol, propylene glycol, glycerol, castor oil, copolymers of propylene oxide and ethylene oxide, and the like; an opacifying agent such as titanium dioxide, iron oxides, and the like.

In one embodiment, the invention provides the process for preparing a sustained release pharmaceutical composition wherein the process comprises the steps of:
mixing alfuzosin, a rate controlling polymer and optionally one or more pharmaceutically acceptable excipients,
granulating the mixture to obtain granules,
drying the granules,
mixing the dried granules with a rate controlling polymer and optionally one or more pharmaceutically acceptable excipients, and
compressing the mixture into a tablet.

In another embodiment, the invention provides the process for preparing a sustained release pharmaceutical composition wherein the process comprises the steps of:
mixing alfuzosin, a rate controlling polymer and optionally one or more pharmaceutically acceptable excipients,
granulating the mixture to obtain granules,
drying the granules,
mixing the dried granules with optionally one or more pharmaceutically acceptable excipients, and
compressing the mixture into a tablet.

In another embodiment, the invention provides the process for preparing a sustained release pharmaceutical composition wherein the process comprises the steps of:
mixing alfuzosin, a rate controlling polymer and optionally one or more pharmaceutically acceptable excipients, and
compressing the mixture into a tablet.

A sustained release composition may include hydroxypropyl methylcellulose in an amount ranging from 10% to 69% by weight, or from 50% to 69% by weight; polyvinylpyrrolidone in an amount ranging from 1% to 25% by weight, 10% to 20% by weight, or from 10% to 15% by weight, lactose in an amount ranging from 5% to 50% by weight, 5% to 30% by weight, or from 10% to 35% by weight, magnesium stearate in an amount ranging from 0.1% to 5% by weight, talc in an amount ranging from 0.1% to 5% by weight and colloidal silicon dioxide in an amount ranging from 0.1% to 5% by weight.

Another sustained release pharmaceutical composition of alfuzosin as described herein exhibits a dissolution of not more than 30% in 4 hour, not more than 45% in 8 hours, not more than 60% in 12 hours, not more than 78% in 20 hours, as measured in 900 ml of 0.01 N HCl using USP Type II apparatus with a paddle speed of 50 rpm at 37±0.5° C.

The term “bioequivalence” as described herein means that there is no significant difference in the rate and extent of bioavailability of the active ingredient between the pharmaceutical composition as described herein and the reference product. The ratios of the log transformed mean values for Cₘₐₓ and AUC for the test and reference product (T/R ratio) is a measure of the bioequivalence between the test and reference product. Values between 80 and 125% for the 90% confidence intervals of these ratios indicate bioequivalence as recommended by the United States Food and Drug Administration.

“Cₘₐₓ” as used herein, means the maximum plasma concentration of the active ingredient (ng/ml), produced after oral ingestion of the composition of alfuzosin or the reference product followed by absorption of active ingredient in plasma.

“AUC” as used herein, means the area under the plasma concentration-time curve over the specified time interval (ng*hr/ml), produced after oral ingestion of the composition of alfuzosin or the reference product followed by absorption of active ingredient in plasma.

The term “reference product” as used herein refers to the compositions containing alfuzosin hydrochloride, which release alfuzosin for an extended period of time of about 12 hours or about 24 hours. The preferred reference product is 10 mg Xatral-XL tablet marketed in Europe, or the 10 mg UroXatral tablet marketed in USA.

The pharmaceutical compositions as described herein may be illustrated by the following examples which are not to be construed as limiting the scope of the invention:

### COMPARATIVE EXAMPLE 1

| | | Quantity | |
|---|---|---|---|
| Ingredients | Functionality | (mg/tablet) | % w/w |
| Alfuzosin hydrochloride | Active ingredient | 10.14 | 2.89 |
| Colloidal silicon dioxide | Glidant | 1.33 | 0.38 |
| Hydroxypropyl | Rate-controlling | 139.00 | 39.71 |
| methylcellulose K | polymer | | |
| 100M CR | | | |
| Polyoxyethylene oxide | Rate-controlling | 45.00 | 12.85 |
| | polymer | | |
| Lactose | Diluent | 112.86 | 32.24 |
| Dibasic calcium phosphate | Diluent | 20.00 | 5.71 |
| anhydrous | | | |
| Polyvinylpyrrolidone K30 | Binder | 15.00 | 4.28 |
| Isopropyl alcohol | Granulating solvent | q.s. | — |
| Talc | Glidant | 2.00 | 0.57 |
| Colloidal silicon dioxide | Glidant | 0.67 | 0.19 |
| Magnesium stearate | Lubricant | 4.00 | 1.14 |
| Total weight | | 349.50 | 100.00 |

Alfuzosin hydrochloride, colloidal silicon dioxide, hydroxypropyl methylcellulose, polyoxyethylene oxide and lactose were sifted and mixed. A solution of polyvinylpyrrolidone was prepared in isopropyl alcohol and the mixture was granulated to obtain granules. The granules were dried, sized, ad mixed with talc, colloidal silicon dioxide and magnesium stearate. The mixture was compressed into the tablets using appropriate tooling.

### COMPARATIVE EXAMPLE 2

| | | Quantity | |
|---|---|---|---|
| Ingredients | Functionality | (Mg/tablet) | % w/w |
| Alfuzosin hydrochloride | Active ingredient | 10.14 | 2.89 |
| Colloidal silicon dioxide | Glidant | 1.00 | 0.29 |
| Hydroxypropyl | Rate-controlling | 245.00 | 70.00 |
| methylcellulose | polymer | | |
| K 100M CR | | | |
| Lactose monohydrate | Diluent | 73.36 | 20.96 |
| Polyvinylpyrrolidone K30 | Binder | 15.00 | 5.00 |
| Isopropyl alcohol | Granulating solvent | q.s. | — |
| Talc | Glidant | 1.00 | 0.28 |
| Magnesium stearate | Lubricant | 3.50 | 1.00 |
| Colloidal Silicon dioxide | Glidant | 1.00 | 0.28 |
| Total weight | | 349.50 | 100.00 |

Alfuzosin hydrochloride, colloidal silicon dioxide, hydroxypropyl methylcellulose and lactose were sifted and mixed. A solution of polyvinylpyrrolidone was prepared in isopropyl alcohol and the mixture was granulated to obtain granules. The granules were dried, sized, and mixed with talc, colloidal silicon dioxide and magnesium stearate. The mixture was compressed into the tablets using appropriate tooling.

### EXAMPLE 3

| | | Quantity | |
|---|---|---|---|
| Ingredients | Functionality | (Mg/tablet) | % w/w |
| Alfuzosin hydrochloride | Active ingredient | 10.20 | 2.91 |
| Colloidal Silicon dioxide | Glidant | 1.00 | 0.29 |
| Hydroxypropyl | Rate-controlling | 139.00 | 39.71 |
| methylcellulose K | polymer | | |
| 100M CR | | | |
| Lactose monohydrate | Diluent | 36.30 | 10.37 |
| Polyvinylpyrrolidone K30 | Binder | 12.00 | 3.43 |
| Isopropyl alcohol | Granulating solvent | q.s. | — |
| Hydroxypropyl | Rate-controlling | 102.50 | 29.29 |
| methylcellulose K | polymer | | |
| 100M CR | | | |
| Polyvinylpyrrolidone K90 | Rate-controlling | 38.50 | 11.00 |
| | polymer | | |
| Talc | Glidant | 2.00 | 0.57 |
| Magnesium stearate | Lubricant | 5.00 | 1.43 |
| Colloidal Silicon dioxide | Glidant | 3.50 | 1.00 |
| Total weight | | 349.50 | 100.00 |

Alfuzosin hydrochloride, colloidal silicon dioxide, hydroxypropyl methylcellulose and lactose were sifted and mixed. A solution of polyvinylpyrrolidone was prepared in isopropyl alcohol and the mixture was granulated to obtain granules. The granules were dried, sized, and mixed with hydroxypropyl methylcellulose, polyvinylpyrrolidone, talc, colloidal silicon dioxide and magnesium stearate. The mixture was compressed into the tablets using appropriate tooling.

### EXAMPLE 4

| | | Quantity | |
|---|---|---|---|
| Ingredients | Functionality | (Mg/tablet) | % w/w |
| Alfuzosin hydrochloride | Active ingredient | 10.20 | 2.91 |
| Colloidal Silicon dioxide | Glidant | 1.00 | 0.29 |
| Hydroxypropyl cellulose | Rate-controlling | 139.00 | 39.71 |
| | polymer | | |
| Lactose | Diluent | 36.30 | 10.37 |
| Polyvinylpyrrolidone K30 | Rate-controlling | 21.00 | 6.00 |
| | polymer | | |
| Isopropyl alcohol | Granulating solvent | q.s. | — |
| Sodium alginate | Rate-controlling | 102.50 | 29.29 |
| | polymer | | |
| Polyvinylpyrrolidone K90 | Rate-controlling | 29.50 | 8.43 |
| | polymer | | |
| Talc | Glidant | 2.00 | 0.57 |
| Magnesium stearate | Lubricant | 5.00 | 1.43 |
| Colloidal Silicon dioxide | Glidant | 3.50 | 1.00 |
| Total weight | | 349.50 | 100.00 |

Alfuzosin hydrochloride, colloidal silicon dioxide, hydroxypropylcellulose and lactose are sifted and mixed. A solution of polyvinylpyrrolidone is prepared in isopropyl alcohol and the mixture is granulated to obtain granules. The granules are dried, sized, and mixed with sodium alginate, polyvinylpyrrolidone, talc, colloidal silicon dioxide and magnesium stearate. The mixture is compressed into tablets using appropriate tooling. The dissolution studies of the compositions as described herein in comparison to the reference product are depicted in Table 1.

TABLE 1
Comparative Dissolution in 900 ml of 0.01 N HCl using USP Type II
Apparatus with a paddle speed of 50 rpm at 37 ± 0.5° C.:

| TIME | COM- | COM- | | |
|---|---|---|---|---|
| IN | PARATIVE | PARATIVE | EXAMPLE | REFERENCE |
| HRS | EXAMPLE 1 | EXAMPLE 2 | 3 | PRODUCT |
| 0 | 0 | 0 | 0 | 0 |
| 1 | 14.70 | 12.7 | 11.3 | 15.3 |
| 3 | 29.40 | 25.8 | 21.8 | 26.7 |
| 4 | 35.50 | 30.9 | 26 | 31.3 |
| 6 | 46.40 | 39.5 | 34 | 39.9 |
| 8 | 56.30 | 47.1 | 41.1 | 50.3 |
| 10 | 64.20 | 54 | 47.4 | 59.1 |
| 12 | 71.50 | 59.9 | 53.3 | 66.9 |
| 16 | 81.90 | 71 | 63.8 | 83.4 |
| 20 | 88.40 | 78.9 | 72.2 | 92 |
| 24 | 91.80 | 86.1 | 80.5 | 94.7 |

The dissolution data indicates that compositions of Comparative Examples 1 and 2 can be expected to be bioequivalent to the reference product. However, as is evident from Tables 2 and 3, when compositions of Comparative Examples 1 and Example 3 were subjected to bioequivalence study with UroXatral as reference product, composition of Comparative Example 1 was not bio-equivalent whereas composition of Example 3 was bio-equivalent to the reference product. Tables 2 and 3 depict the comparison of the pharmacokinetic parameters measured in the bio-equivalent studies. The composition of Example 3 showed log transformed values of Cₘₐₓ, AUC₀₋ₜ and AUC_{0-∞} at 90% confidence interval between 80-125%.

TABLE 2
Comparative Pharmacokinetic Data of Comparative Example 1 and
Reference product (Fed state, n = 12)

| Pharmaco- | Comparative | | | | | Log transformed | |
|---|---|---|---|---|---|---|---|
| kinetic | Example 1 | | Reference | | T/R of | values at 90% CI | |
| Parameters | Mean | S.D. | Mean | S.D. | Mean | Lower | Upper |
| Cₘₐₓ | 14.54 | 8.78 | 7.36 | 2.44 | 1.97 | 126.10 | 236.04 |
| AUC₍₀₋ₜ₎₍ₜ₌₂₄₎ | 168.81 | 90.53 | 96.05 | 58.89 | 1.76 | 119.56 | 249.62 |
| AUC_{(0-∞)} | 180.19 | 86.85 | 92.98 | 32.09 | 1.94 | 120.06 | 250.25 |

TABLE 3
Comparative Pharmacokinetic Data of Example 3 and Reference product
(Fed state, n = 15)

| | | | | | | Log transformed | |
|---|---|---|---|---|---|---|---|
| Pharmacokinetic | Example 3 | | Reference | | T/R of | values at 90% CI | |
| Parameters | Mean | S.D. | Mean | S.D. | Mean | Lower | Upper |
| Cₘₐₓ | 15.30 | 8.50 | 15.20 | 7.88 | 1.01 | 87.74 | 115.73 |
| AUC₍₀₋ₜ₎₍ₜ₌₂₄₎ | 306.60 | 171.71 | 274.84 | 138.20 | 1.11 | 95.91 | 122.20 |
| AUC_{(0-∞)} | 328.73 | 170.07 | 289.13 | 133.19 | 1.14 | 96.36 | 124.79 |

### EXAMPLE 5

| | | Quantity | |
|---|---|---|---|
| Ingredients | Functionality | (mg/tablet) | % w/w |
| Alfuzosin hydrochloride | Active ingredient | 10.00 | 2.86 |
| Hydroxypropyl | Rate-controlling | 139.00 | 39.71 |
| methylcellulose K | polymer | | |
| 100M CR | | | |
| Lactose monohydrate | Diluent | 68.00 | 19.43 |
| Polyvinylpyrrolidone K30 | Binder | 12.00 | 3.43 |
| Colloidal Silicon dioxide | Glidant | 1.00 | 0.29 |
| Isopropyl alcohol | Granulating solvent | q.s. | — |
| Hydroxypropyl | Rate-controlling | 71.50 | 20.43 |
| methylcellulose K | polymer | | |
| 100M CR | | | |
| Polyvinylpyrrolidone K90 | Rate-controlling | 38.50 | 11.00 |
| | polymer | | |
| Talc | Glidant | 2.00 | 0.57 |
| Magnesium stearate | Lubricant | 5.00 | 1.43 |
| Colloidal Silicon dioxide | Glidant | 3.50 | 1.00 |
| Total weight | | 350.00 | 100.00 |

Alfuzosin hydrochloride, colloidal silicon dioxide, hydroxypropyl methylcellulose and lactose were sifted and mixed. A solution of polyvinylpyrrolidone was prepared in isopropyl alcohol and the mixture was granulated to obtain granules. The granules were dried, sized, and mixed with hydroxypropyl methylcellulose, polyvinylpyrrolidone, talc, colloidal silicon dioxide and magnesium stearate. The mixture was compressed into the tablets using appropriate tooling.

TABLE 4
Dissolution profile of Example 5 in 900 ml of 0.01 N HCl using USP
Type II Apparatus with a paddle speed of 50 rpm at 37 ± 0.5° C.:

| TIME IN HRS | EXAMPLE 5 |
|---|---|
| 0 | 0 |
| 1 | 11.3 |
| 3 | 22.7 |
| 4 | 27.8 |
| 6 | 36.6 |
| 8 | 44.4 |
| 10 | 52.0 |
| 12 | 58.7 |
| 16 | 69.8 |
| 20 | 77.9 |
| 24 | 84.5 |

### Bioequivalence Study

Single dose, two way cross over bioequivalence studies were carried out with 70 healthy human volunteers each for both fed and fasted state using extended release tablets comprising 10 mg of alfuzosin, prepared according to Example 5, as test and UroXatral® having alfuzosin 10 mg, as reference. Study was monitored in terms of the pharmacokinetic parameters Cₘₐₓ and AUC.

TABLE 5
Comparative Pharmacokinetic Data of Example 5 and Reference product
(Fed state, n = 70)

| | | | | | | Log transformed | |
|---|---|---|---|---|---|---|---|
| Pharmacokinetic | Example 5 | | Reference | | T/R of | values at 90% CI | |
| Parameters | Mean | S.D. | Mean | S.D. | Mean | Lower | Upper |
| Cₘₐₓ | 12.80 | 4.91 | 12.26 | 4.19 | 1.03 | 95.46 | 110.25 |
| AUC₍₀₋ₜ₎₍ₜ₌₂₄₎ | 208.07 | 88.81 | 200.34 | 90.46 | 1.04 | 93.54 | 116.81 |
| AUC_{(0-∞)} | 219.65 | 83.76 | 210.12 | 89.06 | 1.07 | 96.25 | 117.70 |

TABLE 6
Comparative Pharmacokinetic Data of Example 5 and Reference product
(Fasted state, n = 70)

| | | | | | | Log transformed | |
|---|---|---|---|---|---|---|---|
| Pharmacokinetic | Example 5 | | Reference | | T/R of | values at 90% CI | |
| Parameters | Mean | S.D. | Mean | S.D. | Mean | Lower | Upper |
| Cₘₐₓ | 9.01 | 3.71 | 8.09 | 3.30 | 1.11 | 104.63 | 118.23 |
| AUC₍₀₋ₜ₎₍ₜ₌₂₄₎ | 117.10 | 64.77 | 112.81 | 58.90 | 1.04 | 94.71 | 110.21 |
| AUC_{(0-∞)} | 131.52 | 65.39 | 134.56 | 63.13 | 0.98 | 89.76 | 105.14 |

As can be seen from the data above in Tables 5 and 6, log transformed mean values of Cₘₐₓ, AUC₍₀₋ₜ₎ and AUC_{(0-∞)} at 90% confidence interval for the test and reference product (T/R ratio) are well within the limits of 80-125% as established by the United States Food and Drug Administration for claiming bioequivalence between a test and reference product.

The characteristics of the rate-controlling polymer may be evaluated in the compositions as described herein by measurement of the swelling (S) to erosion (E) ratio (S/E) of the compositions. Not intending to be limited by any theory, the sustained release of alfuzosin from the compositions as described herein is by the diffusion of alfuzosin through the matrix formed by swelling of the rate controlling polymer in contact with the dissolution media or gastro-intestinal fluid, followed by the release of alfuzosin by gradual erosion of the matrix. The comparison between the S/E ratios is given in Table 7:

TABLE 7
Comparative S/E ratios

S/E ratio

| Time | Comparative | | |
|---|---|---|---|
| (in Hours) | Example 1 | Example 3 | Reference product |
| 0 | 1.0 | 1.0 | 2.1 |
| 1 | 1.3 | — | 1.7 |
| 2 | 1.4 | 1.2 | 2.2 |
| 4 | 2.2 | 1.7 | 2.4 |
| 24 | 15.0 | 2.8 | 2.1 |

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A sustained release pharmaceutical composition comprising:
(i) alfuzosin,
(ii) a rate controlling polymer, and
(iii) optionally one or more pharmaceutically acceptable excipients.

2. The composition according to claim 1, wherein the rate controlling polymer comprises polyvinylpyrrolidone in an amount of 10% to 20% by weight of the composition.

3. The composition according to claim 1, wherein the rate controlling polymer comprises hydroxypropyl methylcellulose, hydroxypropyl cellulose, hydroxyethyl cellulose, hydroxypropyl ethylcellulose, methyl cellulose, carboxymethylcellulose, polyvinylpyrrolidone, sodium alginate, xanthan gum, locust bean gum, alginic acid, or methacrylate polymer.

4. The composition according to claim 1, wherein the rate controlling polymer includes hydroxypropyl methylcellulose present in an amount of 35-69% by weight of the composition and polyvinylpyrrolidone present in an amount of 10% to 20% by weight of the composition.

5. The composition according to claim 1, wherein one or more excipients comprise diluent, binder, disintegrant or lubricant.

6. The composition according to claim 1, wherein the composition exhibits a dissolution of not more than 30% in 4 hours, not more than 45% in 8 hours, not more than 60% in 12 hours and not more than 78% in 20 hours as measured in 900 ml of 0.01 N HCl, using USP Type II apparatus, with a paddle speed of 50 rpm, at 37±0.5° C.

7. The composition according to claim 1, wherein the composition is in the form of a tablet.

8. The composition according to claim 1, wherein the rate controlling polymer is present only extragranular, only intragranular or both intragranular and extragranular.

9. A process for preparation of a sustained release pharmaceutical composition, wherein the process comprises:
(i) mixing alfuzosin and optionally one or more pharmaceutically acceptable excipients,
(ii) granulating the mixture of step (i) with a granulating solvent or a solution,
(iii) drying the granules of step (ii),
(iv) mixing the granules of step (iii) with a rate controlling polymer and optionally one or more pharmaceutically acceptable excipients, and
(v) compressing the mixture of step (iv) into a tablet.

10. The process according to claim 9, wherein step (i) comprises a rate controlling polymer.

11. A process for preparation of a sustained release pharmaceutical composition, wherein the process comprises:
(i) mixing alfuzosin, a rate controlling polymer and optionally one or more pharmaceutically acceptable excipients,
(ii) granulating the mixture of step (i) with a granulating solvent or a solution,
(iii) drying the granules of step (ii),
(iv) mixing the granules of step (iii) and optionally one or more pharmaceutically acceptable excipients, and
(v) compressing the mixture of step (iv) into a tablet.

12. The process according to claims 9 or 11, wherein the granulating solvent of step (ii) is isopropyl alcohol, water or mixtures thereof.

13. A sustained release pharmaceutical composition of claim 1 consisting essentially of:
(i) 1-15% by weight alfuzosin,
(ii) 35-69% by weight hydroxypropyl methylcellulose,
(iii) 10-20% by weight polyvinylpyrrolidone, and
(iv) 5-30% by weight lactose.

14. The composition according to claim 13, wherein the composition containing the following:

| | Quantity |
|---|---|
| Ingredients | (mg/tablet) |
| Alfuzosin hydrochloride | 10.00 |
| Hydroxypropyl | 139.00 |
| methylcellulose K 100M CR | |
| Lactose monohydrate | 68.00 |
| Polyvinylpyrrolidone K30 | 12.00 |
| Colloidal Silicon dioxide | 1.00 |
| Isopropyl alcohol | q.s. |
| Hydroxypropyl | 71.50 |
| methylcellulose K 100M CR | |
| Polyvinylpyrrolidone K90 | 38.50 |
| Talc | 2.00 |
| Magnesium stearate | 5.00 |
| Colloidal Silicon dioxide | 3.50 |
| Total weight | 350.00 |

15. A method for the treatment of the signs and symptoms of benign prostatic hyperplasia, wherein the method comprises administering to a patient in need thereof, the composition of claim 1.

16. The composition according to claim 1, wherein the composition exhibits Cₘₐₓ in the range of 3-30 ng/ml and AUC₍₀₋ₜ₎ in the range of 30-550 ng*hr/ml and wherein log transformed values of Cₘₐₓ and AUC₍₀₋ₜ₎ at 90% confidence interval for the test and reference product is within 80-125%.
